(19) United States
(12) Patent Application Publication
(10) Pub. No.: US 2025/0273311 A1
(43) Pub. Date: Aug. 28, 2025

(54) SYSTEM AND ASSOCIATED PROCEDURE FOR IMMEDIATE RISK REDUCTION FOR MEDICAL DEVICES THAT ARE AFFECTED BY A FIELD SAFETY NOTICE (FSN)

(71) Applicant: B. Braun Melsungen AG, Melsungen (DE)

(72) Inventor: Michael Reindl, Melsungen (DE)

(21) Appl. No.: 19/060,092

(22) Filed: Feb. 21, 2025

(30) Foreign Application Priority Data
Feb. 22, 2024 (EP) 24159210.4

(51) Int. Cl. G16H 10/60; G16H 20/10

(52) U.S. Cl. CPC G16H 10/60; G16H 20/10

(57) ABSTRACT

A system for risk mitigation for medical devices affected by an FSN includes an FSN module with a database that stores at least one FSN, unique identifiers of medical devices affected by the respective FSN, and data on risk mitigation measures for the respective FSN. A device management module stores a list of medical devices in use and the unique identifier of the respective medical device. Device management functions are implemented in the device management module. At least one medical device has a unique identifier, control unit and display unit. The FSN module can exchange digital messages with the device management module and/or the respective medical device. The device management module can exchange digital messages with the FSN module and/or with the respective medical device. The respective medical device can exchange digital messages with the FSN module and/or the device management module, and carry out immediate risk reduction measures.

### CROSS-REFERENCE TO RELATED APPLICATION

This application claims priority under 35 U.S.C. § 119 to European Application No. 24 159 210.4, filed on Feb. 22, 2024, the content of which is incorporated by reference herein in its entirety.

### FIELD

The present disclosure relates to a system and an associated method for immediate risk mitigation for medical devices affected by a Field Safety Notice (FSN).

### BACKGROUND

Prior art methods for performing a FSN for a medical device can be characterized by the following steps:
1. start a new FSN
2. identify the medical devices affected and their operators
3. determination of mitigation/corrective measures
4. release of the FSN
5. notify the device operator by letter
6. locate affected medical device(s)
7. remove the medical device from the clinical process
8. applying remedial/corrective measures
9. reuse the medical device
10. quit

The time span for step 5 (notifying the device operator) is in the range of days. The time span for step 6 (locate affected devices) can range from hours to weeks for various reasons.

The medical device fleet can consist of many devices (up to several thousand) that are distributed across different regions, cities, locations, buildings and rooms. The medical devices can also be part of pools and dynamically assigned to different areas of use. The medical devices can be used on the move, e.g. for patient transportation.

Affected medical devices are usually identified by their serial number or other unique ID, which is often not directly visible on the device. As long as the affected medical devices have not yet been localized or are still in use, patients and users may be exposed or affected by the product-related risks highlighted in the FSN.

### SUMMARY

The present disclosure is therefore based on the objective of providing a system and method for rapid risk reduction for medical devices affected by an FSN, which significantly reduces the impact on patients and users caused by the product-related risks highlighted in the FSN.

Regarding the system, this objective is solved according to the present disclosure by a system comprising an FSN module which comprises a database in which at least one FSN and unique identifiers of medical devices affected by the respective FSN as well as a catalog, i.e. essentially a list, of measures for immediate risk reduction are stored and/or can be stored. The system further comprises a device management module in which a list of the operated medical devices and the unique identifier of the respective medical device is stored, and in which a number of device management functions are implemented. The system further comprises at least one medical device, which has a unique identifier, as well as a control unit and a display unit.

In a preferred embodiment, the FSN module and the device management module may be implemented in hardware and/or software in a common computer architecture. For example, the FSN module and the device management module may each be implemented by software and individually and/or shared hardware components. In a further preferred embodiment, the FSN module is designed as an FSN server device and the device management module is designed as a device management device, which are designed to communicate via a data network and have corresponding interfaces for this purpose.

The FSN server module is designed to exchange digital messages with the device management module and/or with the respective medical device, in particular via a data network. The device management module is designed to exchange digital messages with the FSN module and/or with the respective medical device, in particular via a data network. The respective medical device is designed to exchange digital messages with the FSN module and/or with the device management module, in particular via a data network, and to carry out measures for immediate risk reduction.

The present disclosure is based on the consideration that the time between the creation or release of the FSN and the implementation of the necessary measures should be reduced so that, once the risks become known, they can be treated as quickly as possible in the respective medical device by taking appropriate measures.

As has now been recognized, providing digital information about a new FSN to device operators and distributing FSN information to affected medical devices allows immediate implementation of risk mitigation measures, such as displaying a warning message to operators and users, automatic deactivation of affected device functions or other measures. The implementation of these measures is therefore a matter of seconds after the FSN has been approved, whereas conventional methods of executing an FSN usually take longer and carry the risk that not all affected devices are covered.

Advantageously, the device management module has a front end for user interaction. The front end can be designed for execution in a web browser on a desktop PC and/or on a mobile device or in a native app for mobile devices.

The front end is preferably designed to display a list of operated medical devices affected by the FSN. In this way, the device administrator or user of the device management device can obtain an overview of the medical devices affected by the FSN.

Advantageously, the front end is designed to output a warning message for the user. The warning message preferably includes the devices affected by the FSN and/or information of the FSN. In this way, the user or device supervisor of the device management device receives an overview of the number and type of medical devices affected and the type of FSN.

Advantageously, the display unit is designed to display a warning message and/or risk-reducing measure if an FSN is present.

The respective medical device is preferably designed to perform therapeutic and/or patient monitoring and/or diagnostic functions.

In a preferred embodiment, the medical device is an infusion pump or a dialysis machine.

The unique identifier of the medical device is preferably a UDID.

In a preferred embodiment, the digital data is exchanged via a data network, whereby the data network is advantageously the Internet. This allows data to be exchanged between the individual system components (FSN module, device management module, medical device) over any distance. It allows the equipment to be set up and the associated data to be hosted at different locations. The FSN module, the device management module and the respective medical device each have a corresponding interface for this purpose, in particular a wired network interface and/or a WLAN interface.

The FSN module preferably has a push function for an FSN, so that once the FSN has been approved, it can be pushed to the device management module or the medical devices concerned. The device management module and/or the respective medical device can also preferably have a pull function, with the aid of which the FSN module is contacted at regular intervals and an approved FSN can be downloaded.

The device management module and/or the respective medical device is advantageously designed to send a confirmation of receipt to the FSN module when an FSN is received. In this way, it can be ensured that the FSN and the measures for immediate risk reduction have reached the medical device.

The respective medical device is preferably designed to send a confirmation of implementation to the device management module and/or the FSN module or to display a confirmation of implementation on a user interface, in particular a display, after implementation of the measures for immediate risk reduction.

In a preferred embodiment, the device management module is designed to store the status of the implementation of the immediate risk reduction measures associated with an FSN for the respective medical device.

Regarding the computer-implemented method, the above objective is solved according to the present disclosure by a method having the steps mentioned below, the sequence of the method steps not being determined by the sequence of steps given below:
a new FSN is preferably registered or stored in the FSN module. For this purpose, the information relevant to the FSN is preferably entered in the FSN module: an FSN identifier by which the FSN can be uniquely identified, a list of the affected devices or their unique identifiers, data describing the risk mitigation measures (IRMCD-Instant Risk Mitigation Control Data).
the medical devices affected by the FSN are preferably stored in the FSN module. The affected devices are preferably identified outside the described system in a higher-level FSN process by a security officer. The connection between the FSN and the affected devices is established in the FSN server by entering data.
the FSN module prioritizes measures for immediate risk reduction.
the FSN is released in the FSN module. The release of the FSN is part of the higher-level FSN process and is digitally simulated in the FSN module by a corresponding action, for example by a status change.
the FSN module preferably sends a digital message to the device management module containing a list of data on the medical devices affected by the FSN.
the device management module advantageously sends an FSN message to all affected medical devices that are connected and online, which contains the measures for immediate risk reduction.
the medical device in question gives priority to the immediate risk reduction measures.

The measures for immediate risk reduction are preferably mitigating and/or corrective measures.

The immediate risk reduction measures may comprise issuing a warning message to the user with a description of the FSN, in particular the risks for patients and/or users described therein. A machine-readable code, for example a QR code, can also be issued, which contains a link or a reference to immediate risk reduction measures to be carried out by the user.

Another example of a risk-reducing measure is the deactivation or restriction of a device function that causes the risk, such as reducing the backlight of a display to prevent the display unit from failing completely if the brightness is too high.

In a preferred embodiment, the medical device affected by the FSN displays a warning message on the display unit after receiving the FSN message.

Advantageously, the respective medical device notifies the device management module that the mitigating/corrective measures have been carried out.

Preferably, the respective medical device is returned to use after the mitigating/corrective measures have been carried out.

In a preferred embodiment, the FSN module creates a status report on the measures carried out. In this way, it can be traced at a later point in time which measures have been carried out. The status report preferably includes a timestamp or a time interval, the type of action performed and the corresponding unique identifier of the medical device.

The list of data advantageously includes: a unique identifier of the FSN, a list of the unique identifiers of the medical devices and the data on the immediate risk mitigation measures.

In a preferred embodiment of the method, after receiving the digital message, the device management module issues a warning message, in particular as a visual and/or audible warning message and/or as an e-mail message. In this way, the user or operator of the device management module receives comprehensive information about the medical devices affected by the FSN and the imminent measures for immediate risk reduction. The message preferably describes the FSN and displays a list of all affected medical devices.

Advantageously, medical devices that were not connected or online when the FSN message was sent connect to the device management module to query the presence of an FSN message and receive it (if it is present).

The connection to the device management module is preferably established on the basis of a trigger condition, in particular when the device is switched on, when certain device functions are activated, when the network interface is activated, at a certain time of day or on the basis of a cyclical timer. This ensures that the FSN message is retrieved reliably and that FSN messages are retrieved promptly by the medical device.

Advantageously, medical devices that need to be repaired are taken out of service.

The FSN message advantageously includes further measures, in particular repair measures.

The data on the immediate risk mitigation measures, in particular mitigating/corrective measures, are preferably included in the FSN message.

The advantages of the present disclosure lie in particular in the fact that, by training the facilities or devices involved and communicating with them via a data network, the time span between the creation of an FSN and the start of the implementation of the corresponding measures for immediate risk reduction is only a few seconds, since the medical devices affected by the FSN are notified via the data network immediately after the FSN has been created. This greatly reduces the time during which patients or users are still exposed to the risks indicated in the FSN.

### BRIEF DESCRIPTION OF THE DRAWINGS

An embodiment of the present disclosure is explained in more detail below with reference to the drawing figures, which contain a highly schematized representation, of which:

FIG. 1 shows an immediate risk reduction system in a preferred embodiment; and

FIG. 2 shows a method for operating the system according to FIG. 1 in a flow chart in a preferred embodiment.

Identical parts or steps are marked with the same reference signs in both figures.

### DETAILED DESCRIPTION

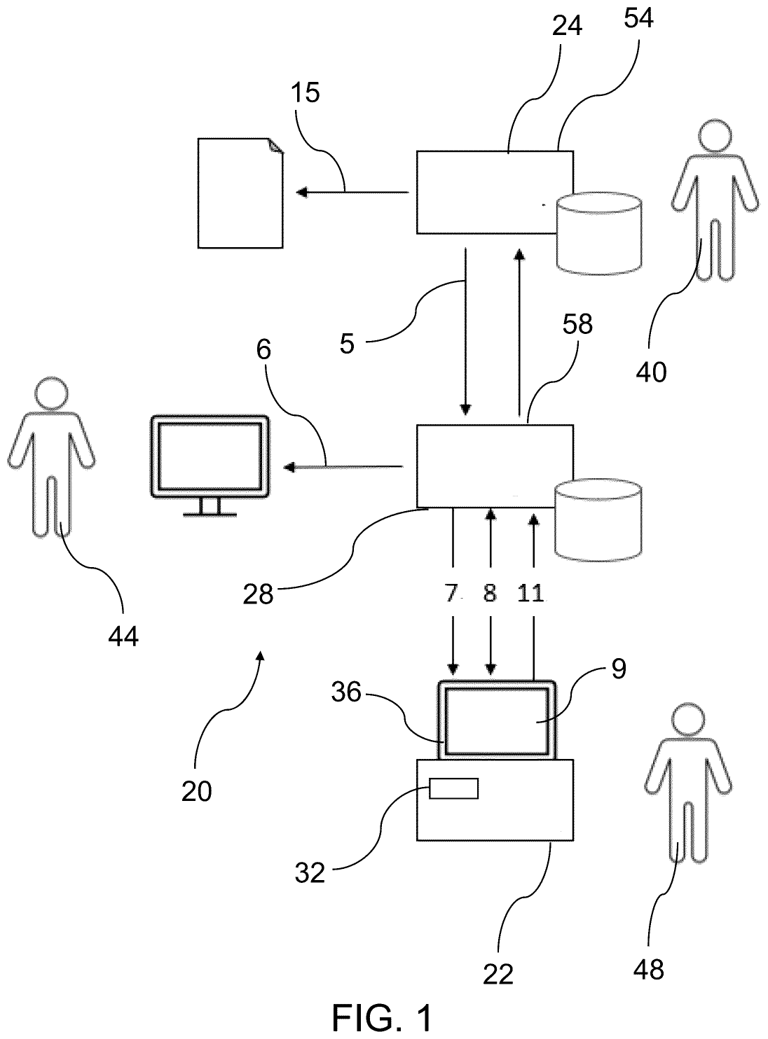

System 20 shown in FIG. 1 comprises an FSN module 54, which in the present case is designed as an FSN server device 24. The FSN server device 24 comprises a software application and/or a service that maintains a database of device-related FSNs. The FSN server facility 24 is preferably hosted by the legitimate manufacturer of a medical device 22 and comprises for each FSN a list of the medical devices 22 concerned, which are identified by their unique identifier, in particular device ID (UDID). For the sake of clarity, only a single medical device 22 is shown in FIG. 1. The FSN server device 24 is operated, in particular configured, by a security officer 40.

For each FSN, the FSN server device 24 comprises a catalog of immediate risk mitigation data or control data (IRMCD) that defines and/or describes immediate risk mitigation measures, such as user alerts and affected device functions

The FSN server device 24 is designed to exchange digital messages with a device management module 58, which in the present case is designed as a device management device 28, and/or the medical device 22 via a data network, in the present case the Internet. The FSN server device 24 and the device management device 28 and the respective medical device 22 are correspondingly designed for data exchange via the Internet and have corresponding interfaces. In particular, these interfaces can be wired or wireless interfaces, especially WLAN.

System 20 comprises the above-mentioned device management device 28, which comprises a software application or service and which manages a list of the medical devices 22 in use. The device management device 28 is adapted to exchange digital messages with the FSN server device 24 and/or the respective medical device 22 via the data network or network. The respective medical device 22 has a UDID (serial number or other unique ID). The device management device 28 stores the associated UDID for each medical device 22.

The device management device 28 is hosted either on site (e.g. in the operator's IT landscape) or in a cloud. It comprises a front-end application that runs in web browsers or on mobile devices and allows a device supervisor 44 to retrieve information and/or interact.

The device management device 28 displays a list of medical devices 22 deployed and used by the operator as needed. It provides various device management functions, such as displaying device status, uploading device configuration data, etc.

System 20 includes the at least one medical device 22 having therapeutic, patient monitoring or diagnostic functions. The respective medical device 22 is software controlled, including one or more microprocessors for executing said software.

The respective medical device 22 further comprises a control unit 32 and a display unit 36 for user interaction (user input, graphical output) for a device user 48. Medical device 22 is adapted to exchange digital messages with the FSN server device 24 and/or the device management device 28 via the data network.

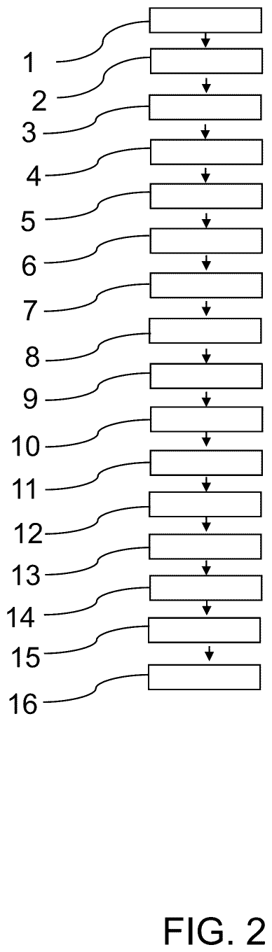

With reference to FIG. 1 and FIG. 2, the functionalities of the system 20 and a method for operating the system 20 in a preferred embodiment are explained below. In FIG. 2, the method is shown in a flowchart. For explanatory purposes, some steps of the method in FIG. 1 are indicated by reference signs.

The computer-implemented method comprises the steps described below.

In a first step 1, a new FSN is registered or stored in the FSN server device 24.

In a second step 2, the medical devices 22 affected by the FSN are stored or saved in the FSN server device 24.

In a third step 3, the measures for immediate risk reduction are defined in the FSN server facility.

In a fourth step 4, the FSN is released or authorized in the FSN server device 24. Steps 1-4 are preferably carried out by a user, in particular the security officer 40.

In a fifth step 5, the FSN server facility 24 uploads the list of unique identifiers of the affected medical devices 22 and the immediate risk mitigation action data to the device management facility 28.

In a sixth step 6, the device management device 28 notifies the device operator by sending a notification of a new FSN and the affected medical devices 22. This notification or alert may be implemented in various ways, such as by displaying a message and/or a list of affected medical devices 22 on the front end of the device management device 28, sending an email notification to the operator, etc.

In a seventh step 7, the device management device 28 sends an FSN message including the immediate risk mitigation action data (IRMCD) to all affected medical devices 22 that are connected and online.

In an eighth step 8, the medical devices 22, which are temporarily disabled or not connected to the network while the device management direction 28 is sending the FSN data, poll for FSN events when they are back online. Medical devices 22 may also use other triggers for polling FSN events, such as start of the medical device 22, activation of defined device functions, activation of the network interface, defined time of day, cyclic timer.

In a ninth step 9, the affected medical device 22 notifies the device user of a new FSN by displaying a warning message on the display or display unit 36 of the medical device 22, including user guidance for further handling of the medical device 22. The message texts, which are preferably part of the immediate risk mitigation action data, may preferably be customized by the safety officer 40

In a tenth step 10, the device performs other immediate risk-mitigating actions, such as deactivating the functions of the affected medical devices 22.

In an eleventh step 11, the medical device 22 acknowledges receipt of the FSN event to the device management device 28, including confirmation of the risk mitigation actions taken. The device management device 28 then forwards it to the FSN server device 24.

In a twelfth step 12, the medical device 22 is removed from the clinical process if physical repair of the medical device 22 is required.

In a thirteenth step 13, further immediate risk reduction measures, i.e. mitigation/corrective measures, are carried out in accordance with the FSN, e.g. an appliance repair.

In a fourteenth step 14, the medical device 22 is reinserted, i.e. put into operation.

In a fifteenth step 15, the FSN service facility 24 prepares a status report on the remedial measures taken; this corresponds to a legal requirement.

In a sixteenth step 16, the process is terminated.

In contrast to the prior art methods described above, only a few seconds elapse between step 4 (approve FSN) and steps 9 and 10, where immediate risk mitigation actions are taken, for the entirety of the affected devices, stopping patient and/or user exposure to the device-related risks highlighted in the FSN. Devices that were missed during the initial escalation of the FSN event will always use the eighth step 8 to receive the latest FSN data when they come back online.

### LIST OF REFERENCE SYMBOLS

1 first step
2 second step
3 third step
4 fourth step
5 fifth step
6 sixth step
7 seventh step
8 eighth step
9 ninth step
10 tenth step
11 eleventh step
12 twelfth step
13 thirteenth step
14 fourteenth step
15 fifteenth step
16 sixteenth step
22 medical device
20 system
24 FSN server device
28 device management device
32 control unit
36 display unit
40 safety officer
44 device supervisor
48 device user
54 FSN module
58 device management module

1. A system for immediate risk mitigation for medical devices affected by a FSN, the system comprising:
an FSN module comprising a database in which at least one FSN and unique identifiers of medical devices affected by the respective FSN as well as a catalog of data on immediate risk mitigation measures for the respective FSN are stored and/or are storable;
a device management module in which a list of medical devices in use and the unique identifier of the respective medical device are stored, and in which a number of device management functions are implemented; and
at least one medical device having a unique identifier and comprising a control unit and a display unit,
the FSN module being designed to exchange digital messages with the device management module and/or with the respective medical device,
the device management module being designed to exchange digital messages with the FSN module and/or with the respective medical device, and
the respective medical device being designed to exchange digital messages with the FSN module and/or with the device management module and to carry out measures for immediate risk reduction.

2. The system according to claim 1, wherein the device management module comprises a front end for user interaction.

3. The system according to claim 2, wherein the front end is designed to display a list of operated medical devices affected by the FSN and/or wherein the front end is designed to output a warning message for a user.

4. The system according to claim 1, wherein the display unit is designed to display a warning message and/or risk-reducing measure if a FSN is present.

5. The system according to claim 1, wherein the respective medical device is adapted to perform therapeutic and/or patient monitoring and/or diagnostic functions.

6. The system according to claim 1, wherein the medical device is an infusion pump or a dialysis machine.

7. The system according to claim 1, wherein the digital data is exchanged via the Internet.

8. A computer-implemented method for operating the system according to claim 1, the computer-implemented method comprising the steps of:
registering a new FSN in the FSN module;
storing the medical devices affected by the FSN in the FSN module;
defining the measures for immediate risk reduction;
enabling the FSN in the FSN module;
sending a digital message from the FSN module to the device management module, the digital message comprising a list of data on the medical devices affected by the FSN;
sending an FSN message from the device management module to all affected medical devices that are connected and online, the FSN message containing data on the measures for immediate risk reduction; and
carrying out the measures for immediate risk reduction in all affected medical devices.

9. The computer-implemented method according to claim 8, wherein all affected medical devices show a warning message on the display unit after receiving the FSN message.

10. The computer-implemented method according to claim 8, wherein all affected medical devices notify the device management module that the measures for immediate risk reduction have been carried out.

11. The computer-implemented method according to claim 8, wherein the list of data comprises:
a unique identifier of the FSN;
a list of the unique identifiers of the medical devices; and
data on immediate risk mitigation measures.

12. The computer-implemented method according to claim 8, wherein, after the step of sending a digital message from the FSN module to the device management module, the device management module outputs a warning message.

13. The computer-implemented method according to claim 12, wherein the warning message comprises at least one of:
a visual warning message;
an acoustic warning message; or
an e-mail message.

14. The computer-implemented method according to claim 8, wherein medical devices that are not connected or online when the FSN message is sent connect to the device management module to query a presence of an FSN message and receive the FSN message.

15. The computer-implemented method according to claim 14, wherein connection to the device management module takes place based on a trigger condition.

16. The computer-implemented method according to claim 15, wherein the trigger condition comprises one of:
switching on of the medical device;
activation of certain device functions; or
activation of a network interface,
at a certain time of day, based on a cyclic timer.

17. The computer-implemented method according to claim 8, further comprising the step of taking medical devices that require repair out of service.

18. The computer-implemented method according to claim 8, wherein the FSN message contains further measures.

19. The computer-implemented method according to claim 18, wherein the further measures comprise repair measures.

20. The computer-implemented method according to claim 8, wherein the data on the measures for immediate risk reduction is included as instant risk mitigation control data in the FSN message.
